(19) United States
(12) Patent Application Publication
(10) Pub. No.: US 2010/0168685 A1
(43) Pub. Date: Jul. 1, 2010

(54) Serially Dispensable Antiseptic Wet Wipes Moistened with Tincture of Green Soap

(76) Inventor: Randall E. Drown, Grand Rapids, MN (US)

Correspondence Address: Michael A. Kondzella, Suite 112, 10650 County Road 81, Maple Grove, MN, 55369, US

(21) Appl. No.: 12/344,491

(22) Filed: Dec. 27, 2008

(51) Int. Cl. A61M 35/00; A61K 31/19; B65D 83/00; A61P 31/02

(52) U.S. Cl. 604/289; 514/557; 221/46

(57) ABSTRACT

An apparatus and method of use are disclosed for rapidly and repeatedly cleaning a skin area during tattooing or body piercing. The apparatus includes a plurality of serially dispensable, antiseptic wet wipes pre-moistened with “tincture of green soap” solution. It eliminates any need to dilute a concentrated green soap solution or clean a spray bottle, and allows a tattoo artist to quickly and conveniently obtain and use a wet wipe of the invention in a controlled and consistent manner using only one hand, thereby avoiding interruption of the procedure and contamination of the wet wipe. The wet wipes and/or green soap can be medically approved, and the wipes can be disposable and/or made from non-woven paper, with a preferable size of 19.5 by 17.0 centimeters. The wet wipes can be interleaved or formed in a continuous sheet with perforated separations, and can be folded, or configured in a roll.

### FIELD OF THE INVENTION

The invention generally relates to the application of tattoos, and more specifically to cleaning and reducing infection during the tattoo application process.

### BACKGROUND OF THE INVENTION

Application of a tattoo requires penetration of the skin repeatedly over an extended area so as to inject the tattooing ink. So as to avoid infection, the area to be tattooed must first be cleaned and disinfected. In addition, as the tattooing proceeds, excess ink and small quantities of blood typically accumulate on the skin, and it is therefore necessary to repeatedly clean and re-disinfect the area of the skin where the tattoo is being applied.

The most commonly used method for cleaning an area of skin to which a tattoo is being applied is to moisten a paper towel with a cleansing and disinfecting solution commonly referred to as “Tincture of Green Soap”, or more simply referred to as “Green Soap,” and then to wipe the area with the moistened paper towel. The green soap can be either poured or sprayed onto the paper towel, for example.

Green Soap is a translucent, yellowish liquid soap made from vegetable oils. Chemically, “Green soap” is a potassium soap made by the saponification of suitable vegetable oils, excluding coconut oil and palm kernel oil, without the removal of glycerin. Green soap is typically supplied as a concentrate, and before use must be properly diluted with the correct amount of water, according to the manufacturer's instructions. However, diluting the green soap with water is time consuming and inconvenient. It can also be error-prone, since accidentally or unknowingly mixing green soap with the wrong quantity of water, can result in reduced effectiveness and potential health risks. Also, water that contains particulate impurities can contaminate the skin, and/or water that is too “hard” (i.e. has a high ion content) can negatively impact the safety and/or effectiveness of the green soap solution.

As an alternative to moistening the paper towel with green soap, a spray bottle can be used to apply the green soap solution directly to the tattoo application area of the skin before wiping the skin with a dry paper towel. However, cleaning of the spray bottle between uses is time-consuming and inconvenient, and improper cleaning can lead to introduction of contaminants, cross contamination between tattoo recipients, and associated health risks.

Furthermore, the application of green soap using paper towels, with or without a spray bottle, requires the use of both hands. Therefore, if the tattoo artist does not have an assistant, the tattoo artist is required to suspend application of the tattoo so as to clean and disinfect the area of the skin where the tattoo is being applied. As a result, the tattooing process is interrupted and prolonged, and the tattoo artist is discouraged from cleaning the application area as frequently as might be needed. In addition, portions of the paper towel that are not disinfected by the green soap and are therefore not sterile can come into contact with the tattoo wound area and possibly introduce infectious agents.

Wet wipes impregnated with green soap are available in individually-sealed foil packets, the foil packets often being further packaged in a box. However, it is necessary to use a substantial number of such wet wipes rapidly in sequence during a tattoo procedure. Consequently, this approach is expensive, and it is difficult and inconvenient to handle and open each packet as rapidly as needed during the tattooing procedure. If a tattoo practitioner or an assistant thereof opens all of the packets before the tattoo procedure begins, the wet wipes may at least partially dry out and/or become contaminated before they are used, thereby increasing the danger of a skin infection.

### SUMMARY OF THE INVENTION

An apparatus for rapidly, repeatedly, and inexpensively cleaning a skin area during a tattooing or body piercing procedure is claimed. Wet wipes are pre-moistened with a cleansing and disinfecting soap solution, preferably a tincture of green soap solution, which is suitable for use during application of a tattoo or during body piercing. Each wet wipe is uniformly saturated over the entire wipe at an optimal level of saturation. The wipes are then rapidly and serially dispensed from a dispenser. A tattoo artist is thereby enabled to rapidly and conveniently obtain a sterile, green-soap-moistened wet wipe whenever needed, using only one hand. Any need to pre-mix the green soap solution is eliminated. Pre-moistening of the wet wipes ensures that all portions of each wet wipe are disinfected before use. Interruption of the tattoo application process is minimized, and the skin area is cleaned and disinfected more frequently and consistently.

Also, the cost of using the present invention is significantly reduced as compared to the prior art, being typically $40 per month for a full-time tattoo artist, including labor, as compared to $170 per month using paper towels moistened with green soap, and $340 per month using individual, pre-sealed packets.

In preferred embodiments, the cleansing and disinfecting soap solution and/or the wet wipes are approved for medical use. In further preferred embodiments, each wet wipe is a non-woven paper product. In some preferred embodiments, the wet wipes are interleaved in the dispenser or formed in a continuous sheet with perforated separations. The wet wipes can be folded in the dispenser, or configured as a roll.

One general aspect of the present invention is an apparatus for rapidly and repeatedly cleaning a skin area during a tattooing or body piercing procedure. The apparatus includes a dispenser with a portion capable of serially dispensing wet wipes and a plurality of wet wipes contained within the dispenser, each wet wipe being moistened with a cleansing and disinfecting soap solution that is suitable for use during a body piercing or application of a tattoo.

In preferred embodiments the cleansing and disinfecting soap solution includes tincture of green soap, and in some of these embodiments, the tincture of green soap is approved for medical applications.

In certain preferred embodiments the dispenser includes a top portion with an opening through which the wet wipes can be serially dispensed. In some of these embodiments the opening includes a gripping mechanism that is able to grip a wet wipe so as to facilitate separation of a perforation. And in other of these embodiments the wet wipes are serially dispensable such that dispensing of a first wet wipe causes a second wet wipe to be drawn partially through the opening so as to be ready to be dispensed.

In some embodiments the dispenser includes a cap that is able to seal the dispenser when the dispenser is not in use. In various preferred embodiments the cleansing and disinfecting soap is approved for medical applications. In other preferred embodiments the wet wipes are made from non-woven paper.

In preferred embodiments the wet wipes are one of folded in a stack and arranged in a roll. In other preferred embodiments the wet wipes are interleaved. And in still other preferred embodiments, the wet wipes are part of an elongated sheet and are separated by perforations.

In various preferred embodiments the medical cleansing soap is a mixture of 16 parts water to 1 part tincture of green soap. And in some of these embodiments the water is purified by reverse osmosis.

Another general embodiment of the present invention is a method for rapidly and repeatedly cleaning a skin area during a tattoo procedure. The method includes obtaining a wet wipe moistened with a tincture of green soap solution from a dispenser capable of serially and rapidly dispensing wet wipes, wiping the skin area with the wet wipe, and repeating as needed.

In preferred embodiments, the dispenser includes a top portion with an opening through which the wet wipes can be serially dispensed. In other preferred embodiments the tincture of green soap is approved for medical applications. In certain preferred embodiments the wet wipes are serially dispensable such that dispensing of a first wet wipe causes a second wet wipe to be drawn partially through the opening so as to be ready to be dispensed.

In various preferred embodiments wet wipes are configured in the dispenser as one of a folded stack and a roll. And in still other preferred embodiments wet wipes in the dispenser are one of interleaved and separated by perforations.

### BRIEF DESCRIPTION OF THE DRAWINGS

The invention will be more fully understood by reference to the detailed description, in conjunction with the following figures, wherein:

FIG. 1 is a perspective drawing of a preferred embodiment in which the wet wipes are held in a round dispenser, folded in a continuous stack, separated by perforations, and dispensed through a round hole with a flip-top lid;

FIG. 2 is a perspective drawings of a preferred embodiment in which the wet wipes are held in a round dispenser that is weighted at the bottom, the wipes being configured in a continuous roll and dispensed through a rectangular opening that includes a cutting edge;

FIG. 3 is a perspective drawing of a preferred embodiment in which the wet wipes are held in a rectangular dispenser, configured in a continuous roll held by a spindle, and dispensed through a rectangular opening that includes a cutting edge;

FIG. 4A is a perspective drawing of a preferred embodiment in which the wet wipes are held in a wide rectangular dispenser with a flip-open lid attached at one edge to the dispenser lid, shown with the flip-open lid closed;

FIG. 4B is a perspective drawing of the preferred embodiment of FIG. 4A, shown with the flip-open lid opened and revealing a wet wipe ready for dispensing through a wide slot;

FIG. 4C is a perspective drawing of an embodiment similar to the embodiment of FIG. 4B, in which the wet wipes are folded within the dispenser and are accessed without being drawn through an opening;

FIG. 5A is a perspective drawing of an embodiment that includes a dispensing tub that can be refilled with wet wipes; and

FIG. 5B is a perspective view of a refill pack of wet wipes compatible with the embodiment of FIG. 5A.

### DETAILED DESCRIPTION OF PREFERRED EMBODIMENTS

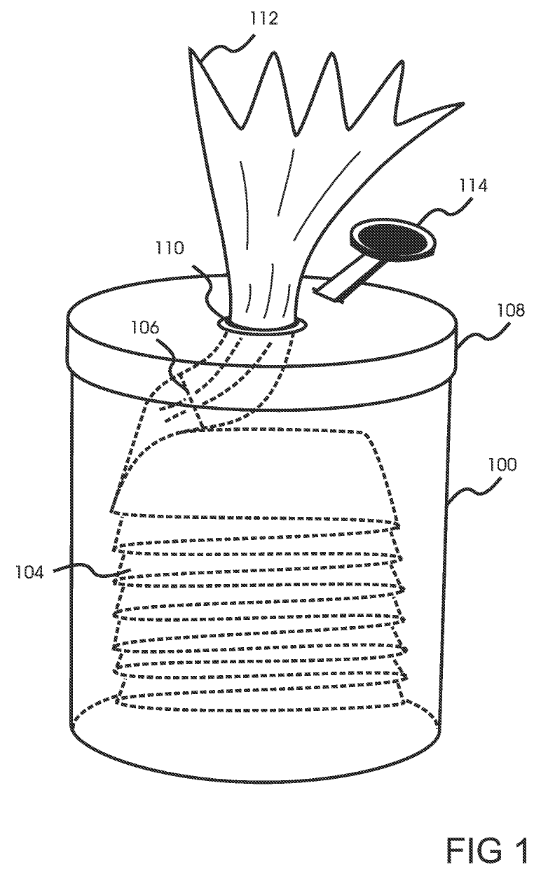

With reference to FIG. 1, the present invention is an apparatus for rapidly and repeatedly cleaning a skin area during one of a tattooing and body piercing procedure. A dispenser 100 is filled with a plurality of wet wipes 104 that are pre-moistened or “converted” with a cleansing and disinfecting soap solution that is suitable for use during application of a tattoo or during body piercing. In the embodiment of FIG. 1, the cleansing and disinfecting soap is a tincture of green soap diluted as a mixture of 16 parts purified water with 1 part tincture of green soap, the water being purified by reverse osmosis, deionization, or distilling.

In preferred embodiments, the wet wipes are approved for medical use, and in some preferred embodiments the wet wipes are non-woven paper wet wipes. In other embodiments the wipes are made from woven paper, cloth, chamois, cheesecloth, cotton balls, gauze pads, or any other absorbent, disposable material. In various embodiments, the wet wipes are rectangular with dimensions of 19.5 centimeters by 17.0 centimeters (7.7 inches by 6.7 inches).

In the embodiment of FIG. 1, the wet wipes 104 are configured in a continuous sheet that is folded and separated by perforations 106. The top 108 of the dispenser includes an opening 110 through which an end 112 of a wet wipe can extend, so as to be grasped and pulled from the dispenser, thereby drawing a fresh wet wipe partially through the opening 110 in preparation for dispensing. The embodiment of FIG. 1 also includes a cap 114 attached to the top 108 of the dispenser, the cap 114 being in a “flip-arm” configuration that allows the cap 114 to be easily installed over the opening 110 so as to seal the dispenser 100 when it is not in use, and easily “flipped” open when the dispenser 100 is to be used. In similar embodiments, the wet wipes 104 are configured as separate sheets that are interleaved, such that removal of a wet wipe 104 causes a fresh wet wipe 104 to be pulled partially through the opening 110 in preparation for dispensing.

In some preferred embodiments, the tincture of green soap is supplied, for example, by Cosco Soap and Detergent Co. In further preferred embodiments, the cleansing and disinfecting soap solution 102 is a mixture of 16 parts water treated by reverse osmosis for purity, combined with 1 part tincture of green soap. The wet wipes 104 can be dispensed from a plastic, injection-molded, push-button/flip-open dispenser 100 with a rear hinged lid 114. The size of the dispenser 100 is preferably 8 inches long by 4¼ inches tall by 5 inches wide, and the dispenser lid 114 is preferably 5 inches long by 2½ inches wide with a dispenser opening 110 of 2⅝ inches, cut in a serpentine design. In preferred embodiments, the dispenser 100 is a 60 count plastic tub dispenser, shrink wrapped and factory sealed before use.

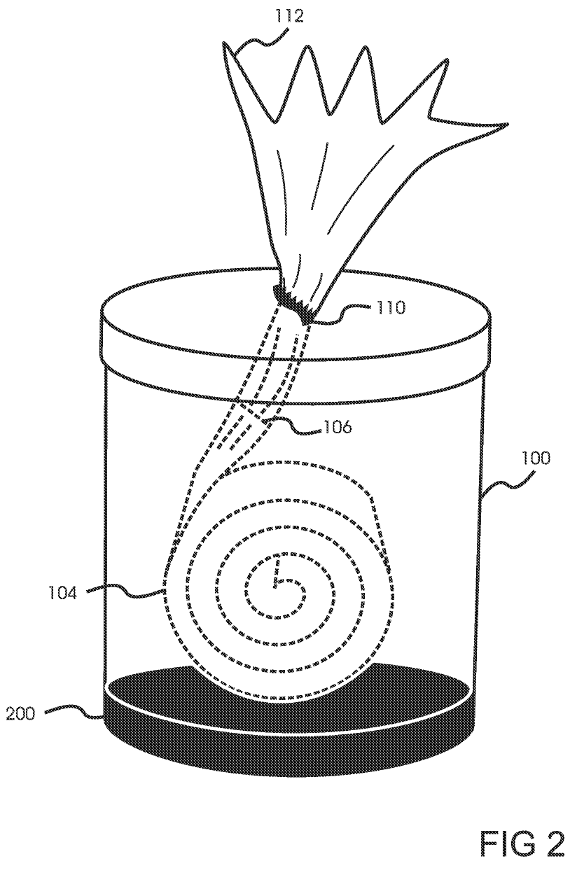

FIG. 2 illustrates an embodiment similar to the embodiment of FIG. 1, except that the wet wipes 104 are configured as a roll, and the opening 110 is a slot configured in a serpentine shape with teeth that can grip a wet wipe and hold it in place to facilitate tearing of a perforation between wet wipes.

For known wet-wipe dispensers, a dispenser can be inadvertently lifted, moved, or overturned during an attempt to remove a wet wipe, especially when the dispenser is nearly empty. This can make it necessary to use two hands to obtain a wet wipe, thereby disrupting any procedure that is in progress. Also, it can be inconvenient to return the dispenser to its desired location, and damage to nearby items can occur, for example if the dispenser is inadvertently moved such that it knocks other items to the floor. Contamination of one or more wipes can also occur if, for example, the dispenser itself is knocked to the floor or brought into contact with a non-sterile object. Therefore, in the embodiment of FIG. 2 the dispenser 100 includes a weight 200 at the bottom for increased stability. The weight can be a weighted disk, a weighted cross, a weighted bar, or other weighted shape, and can be made from lead, iron, sand, steel, or other heavy material. In other preferred embodiments the dispenser is attached to a wall, to the floor, or to a tattoo workstation using hook-and-loop fastening, glue, screws, or by any other fastening means known in the art.

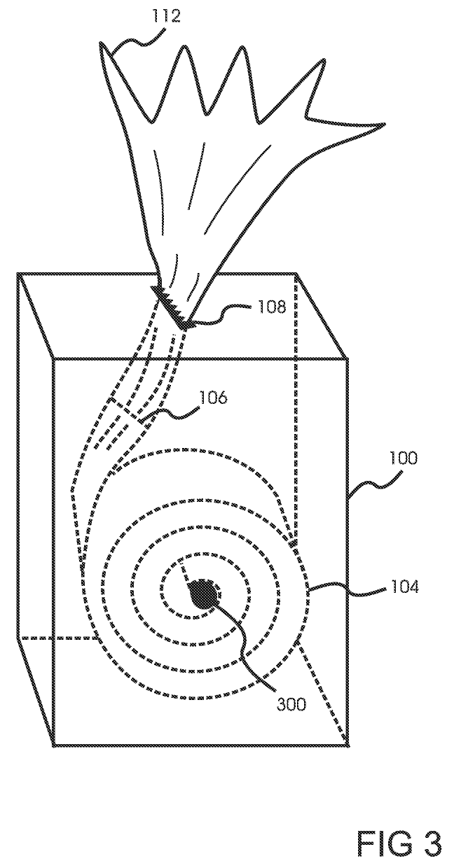

FIG. 3 illustrates a preferred embodiment in which the dispenser 100 is rectangular in shape, and the wet wipes 104 are configured in a roll that is supported by a spindle 300. In this embodiment the opening 108 is a rectangular slot with wet wipe-gripping or cutting teeth.

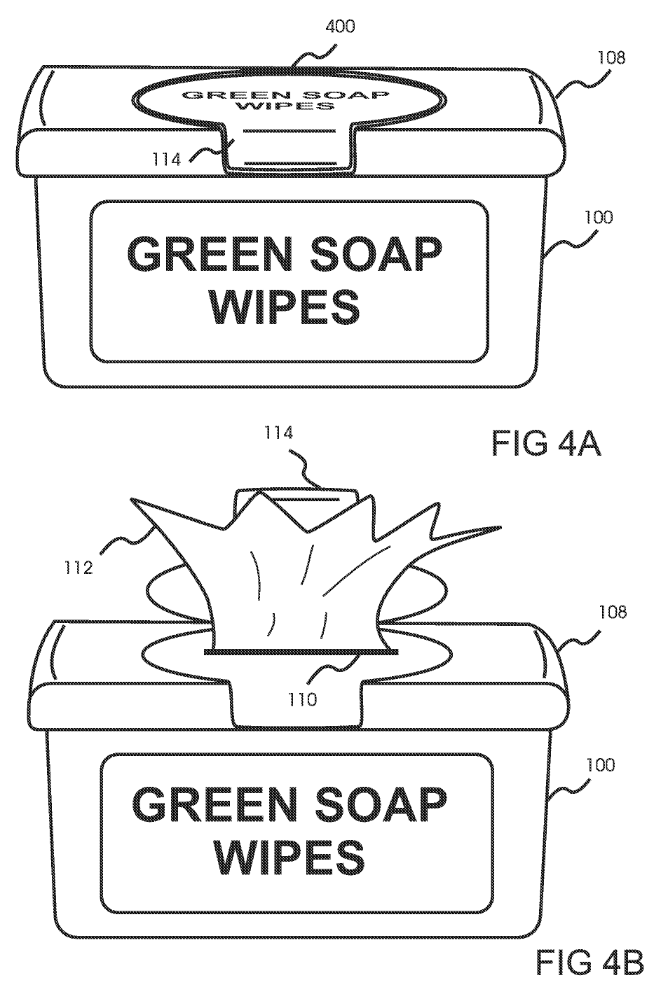

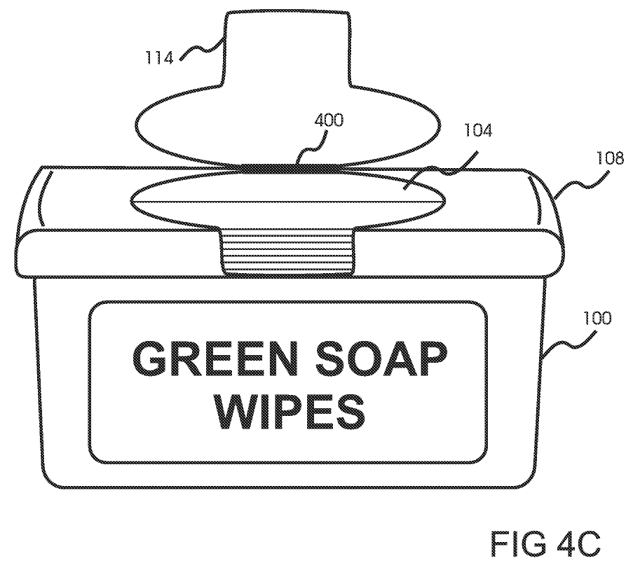

FIG. 4A illustrates a preferred embodiment in which the dispenser 100 has a wide rectangular shape with a large and wide cap 114 attached to the cover 108 of the dispenser 100 at one edge by a plastic hinge 400. FIG. 4B illustrates the embodiment of FIG. 4A with the lid open, showing a wet wipe partially drawn through a wide slot opening 110. FIG. 4C illustrates a preferred embodiment similar to FIG. 4B, in which folded wet wipes 104 are directly accessible through the open lid, without need to pass through a small opening 110.

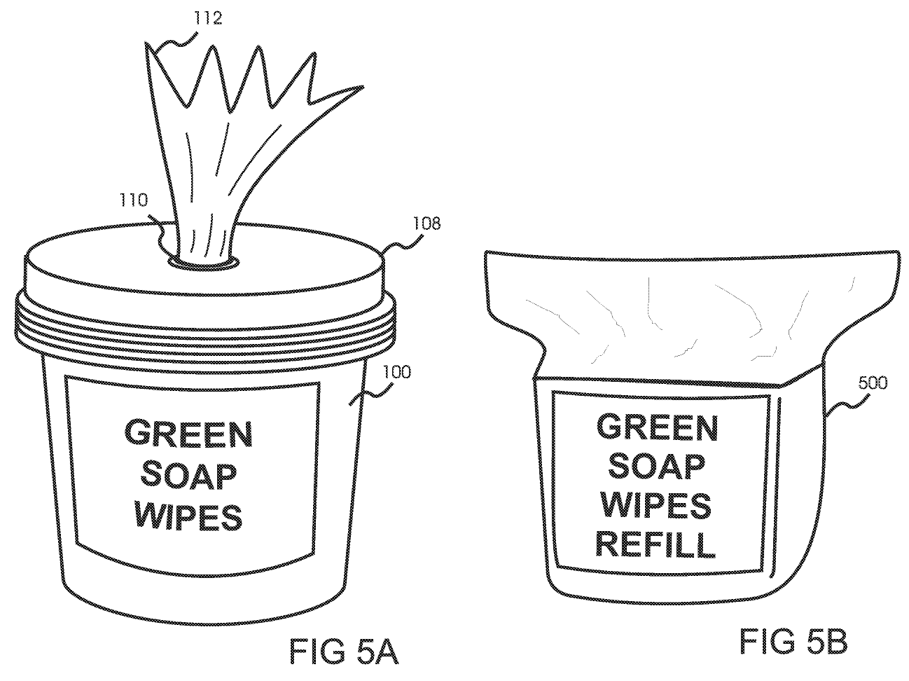

FIG. 5A illustrates an embodiment similar to the embodiment of FIG. 1, in which wet wipes 104 are contained in a round, tub-like dispenser 100 that can be refilled and reused. A refill pack 500 of green-soap moistened wet-wipes 104 is illustrated in FIG. 5B.

Other modifications and implementations will occur to those skilled in the art without departing from the spirit and the scope of the invention as claimed. Accordingly, the above description is not intended to limit the invention except as indicated in the following claims.

What is claimed is:

1. An apparatus for rapidly and repeatedly cleaning a skin area during one of a tattooing and a body piercing procedure, the apparatus comprising:
a dispenser having a portion capable of serially dispensing wet wipes; and
a plurality of wet wipes, contained within the dispenser, each wet wipe being moistened with a cleansing and disinfecting soap solution that is suitable for use during at least one of body piercing and application of a tattoo.

2. The apparatus of claim 1, wherein the cleansing and disinfecting soap solution includes tincture of green soap.

3. The apparatus of claim 2, wherein the tincture of green soap is approved for medical applications.

4. The apparatus of claim 1, wherein the dispenser includes a top portion with an opening through which the wet wipes can be serially dispensed.

5. The apparatus of claim 4, wherein the opening includes a gripping mechanism that is able to grip a wet wipe so as to facilitate separation of a perforation.

6. The apparatus of claim 4, wherein the wet wipes are serially dispensable such that dispensing of a first wet wipe causes a second wet wipe to be drawn partially through the opening so as to be ready to be dispensed.

7. The apparatus of claim 1, wherein the dispenser includes a cap that is able to seal the dispenser when the dispenser is not in use.

8. The apparatus of claim 1, wherein the cleansing and disinfecting soap is approved for medical applications.

9. The apparatus of claim 1, wherein the wet wipes are made from non-woven paper.

10. The apparatus of claim 1, wherein the wet wipes are one of folded in a stack and arranged in a roll.

11. The apparatus of claim 1, wherein the wet wipes are interleaved.

12. The apparatus of claim 1, wherein the wet wipes are part of an elongated sheet and are separated by perforations.

13. The apparatus of claim 1, wherein the medical cleansing soap is a mixture of 16 parts water to 1 part tincture of green soap.

14. The wet wipe dispenser of claim 13, wherein the water is purified by reverse osmosis.

15. A method for rapidly and repeatedly cleaning a skin area during one of a tattooing and a body piercing procedure, the method comprising:
obtaining a wet wipe moistened with a tincture of green soap solution from a dispenser capable of serially and rapidly dispensing wet wipes;
wiping the skin area with the wet wipe; and
repeating as needed.

16. The method of claim 15, wherein the dispenser includes a top portion with an opening through which the wet wipes can be serially dispensed.

17. The method of claim 15, wherein the tincture of green soap is approved for medical applications.

18. The method of claim 15, wherein the wet wipes are serially dispensable such that dispensing of a first wet wipe causes a second wet wipe to be drawn partially through the opening so as to be ready to be dispensed.

19. The method of claim 15, wherein wet wipes are configured in the dispenser as one of a folded stack and a roll.

20. The method of claim 15, wherein wet wipes in the dispenser are one of interleaved and separated by perforations.
